SEQUENCE LISTING (3 sequences)

SEQ ID NO: 1 - DNA, 25 bp, Artificial Sequence
  Description of Artificial Sequence Synthetic primer
caccaagaaaccaattaatgtagca

SEQ ID NO: 2 - DNA, 35 bp, Artificial Sequence
  Description of Artificial Sequence Synthetic primer
ctatagagaattatgtttgttgtatatatcggtag

SEQ ID NO: 3 - protein, 6 aa, Artificial Sequence
  Description of Artificial Sequence Synthetic 6xHis tag
HHHHHH

(19) United States
(12) Patent Application Publication
(10) Pub. No.: US 2024/0239821 A1
(43) Pub. Date: Jul. 18, 2024

(54) N-ACYL FOSMIDOMYCIN PRODRUG ANALOGS AS NOVEL ANTIINFECTIVE AGENTS

(71) Applicant: THE GEORGE WASHINGTON UNIVERSITY, Washington, DC (US)

(72) Inventors: Cynthia S. DOWD, Washington, DC (US); Kenneth HEIDEL, Washington, DC (US); Audrey R. Odom JOHN, Philadelphia, PA (US)

(21) Appl. No.: 18/559,017

(22) PCT Filed: May 6, 2022
(86) PCT No.: PCT/US2022/027998

§ 371 Date: Nov. 3, 2023

Related U.S. Application Data
(60) Provisional application No. 63/185,197, filed on May 6, 2021.

(51) Int. Cl. C07F 9/40; A01N 57/04; A01P 13/00; C07F 9/38

(52) U.S. Cl. CPC C07F 9/4015; A01N 57/04; A01P 13/00; C07F 9/3826

(57) ABSTRACT

The present disclosure relates to novel compounds useful as antimicrobial agents. The present disclosure also relates to processes for their preparation, pharmaceutical compositions comprising them, and to their use in methods for treating or preventing microbial infections caused by parasites or bacteria, such as, for example, Plasmodium falciparum or related Plasmodium parasite species, Mycobacterium tuberculosis or related Mycobacterium bacteria species, S aureus, and ESKAPE pathogens.

### CROSS REFERENCE TO RELATED APPLICATIONS

This application claims the benefit of U.S. Provisional Application No. 63/185,197, filed May 6, 2021, the entire contents of which are hereby incorporated by reference.

### STATEMENT REGARDING FEDERALLY SPONSORED RESEARCH OR DEVELOPMENT

At least some aspects of this invention were made with Government support from National Institutes of Health under Grant No. A1 123433. The Government may have certain rights in this invention.

### FIELD OF THE INVENTION

The present invention relates to novel compounds useful as antimicrobial agents. The present invention also relates to processes for their preparation, pharmaceutical compositions comprising them, and to their use in methods for treating or preventing microbial infections caused by parasites or bacteria, such as, for example, Plasmodium falcipavrum or related Plasmodium parasite species, Mycobacterium tuberculosis or related Mycobacteriun bacteria species, Saureus, and ESKAPE pathogens, including drug resistant strains of such microorganisms. The compounds described herein may also be useful as herbicides.

### BACKGROUND OF THE INVENTION

Despite intense efforts in drug development and aggressive vector control programs, malaria remains a formidable challenge to public health. According to recent estimates, malaria causes 212 million clinical cases and more than 429,000 deaths each year, predominately in young children living in sub-Saharan Africa. While 5 species of Apicomplexan parasites of the genus Plasmodium cause human malaria, Plasm odium falciparum is the most deadly. Due to pervasive drug resistance, P. falciparum treatment has become increasingly dependent on a single class of compounds, the artemisinins. However, there is substantial evidence to suggest that the effectiveness of artemisinin combination therapies (ACTs) is waning, and as such, global malaria control efforts are threatened. The rapid increase in multidrug-resistant parasites combined with a chronic under-investment in drug discovery has severely limited existing therapies. As only a few new antimalarial agents are in the clinical pipeline, identification of novel drug targets is essential.

The methylerythritol phosphate (MEP) pathway of isoprenoid biosynthesis is an unexploited drug target present in most eubacteria and apicomplexan protozoa. In P. falcipavrum, the MEP pathway enzymes are apicoplast-localized, and data suggest that isoprenoid precursor biosynthesis is the only essential function of the plastid organelle in blood-stage parasites. The pathway begins with the condensation of pyruvate and glyceraldehyde-3-phosphate and then proceeds through a series of enzymatic reactions to produce isopentenyl pyrophosphate (IPP) and dimethylallyl diphosphate (DMAPP), which are used to synthesize downstream products. The enzymes of the MEP pathway are essential, as isoprenoids are required for numerous cellular processes including aerobic respiration, membrane stability, and protein prenylation. Importantly, humans employ an alternate route for isoprenoid generation, using instead the mevalonate pathway whose components lack similarity to MEP pathway enzymes. Due to the essentiality of the MEP pathway in P. falciparum (FIG. 1) and the absence of mammalian homologs, compounds that would specifically inhibit enzymes in the pathway are paramount.

The first committed enzyme of the MEP pathway is catalyzed by 1-deoxy-D-xylulose-5-phosphate reductoisomnerase (Dxr/IspC; EC 1 1.1.267), and considerable efforts have been made to effectively target the enzyme. Dxr catalyzes the reductive isomerization of 1-deoxy-D-xylylose 5-phosphate (DOXP) to 2-C-methyl-D-erythritol 3-phosphate (MEP), using a divalent cation (Mg²⁺, Mn²⁺, or Co²⁺) and NADPH as a cofactor. Chemical inhibition of Dxr in blood-stageP. Ja/ciparm depletes cellular MEP metabolites, and ultimately kills the parasites. Moreover, genetic disruption of the Dxr locus in P. falciparum (PF3D7 1467300) is only feasible if cultures are artificially supplemented with downstream isoprenoids. Further, Dxr is druggable, contains a high flux-control coefficient, and is one of only seven antimalarial targets that have been clinically validated.

Mycobacterium tuberculosis (Mtb) is the causative agent of tuberculosis. Two mechanisms are known for the biosynthetic production of isoprenoid units: the mevalonate pathway found in mammals and plants, and the nonmevalonate pathway found in most bacteria. There are no human homologues for the enzymes of the nonmevalonate pathway and each enzymatic reaction is vital to the survival of bacteria. These enzymes are thus prospective targets for therapeutic intervention of M tuberculosis. Dxr is essential for the growth of Mtb. Current anti-TB drugs do not target the nonmevalonate pathway, so Dxr inhibition would be a new mechanism of action.

Fosmidomycin (1a), isolated from Streptomyces lavendulae, is a potent inhibitor of P, falciparum DXR (IC50=0.034 uM). FR900098 (1b), the N-acetyl analog of fosmidomycin isolated from Streptomyces rubellomurinus, is roughly equipotent to fosmidomnycin (P. falciparum DXR IC₅₀=0.024 μM). While these two natural products have submicromolar inhibition of P. falciparum growth (IC₅₀=0.09-0.35 μM), their use as a single drug therapy is limited by low bioavailability, short serum half-life, and malaria recrudescence.

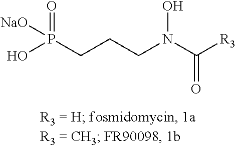

There is therefore a need for new Dxr inhibitors to combat microbial infections caused by, for example, P. falciparum malaria and ML tuberculosis.

### SUMMARY OF THE INVENTION

In one aspect, the present invention relates to a compound of formula (I)

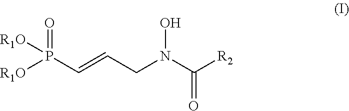

or a tautomer thereof, stereoisomer thereof, prodrug thereof, or pharmaceutically acceptable salt thereof,

wherein

each R₁ is, independently, H, C₁₋₆ alkyl, C₁₋₆ haloalkyl, C₃₋₆ cycloalkyl, —(CR^{a}R^{b})ₘ-aryl —(CR^{a}R^{b})ₘ—O(C═O)—C₁₋₆ alkyl (e.g., —CH₂—O—C(═O)—C(CH₃)₃), —(CR^{a}R^{b})ₘ—O(C═O)—C₃₋₆ cycloalkyl, —(CR^{a}R^{b})ₘ—O(C═O)-aryl (e g., —CH₂—O—C(═O—C₆H₅), —(CR^{a}R^{b})—O(C═O)O—C₁₋₆ alkyl, or —(CR^{a}R^{b})ₘ—O(C═O)O—C₃₋₆ cycloalkyl, —NH₄, —N(alkyl)₄ or —N(aryl)₄, wherein the atom at the left is attached to the oxygen atom;

R² is aryl (e.g., phenyl, naphthyl) or heteroaryl (e.g., pyridinyi);

each of R^{a} and R^{b} is, independently, H, halogen, or C₁₋₄ alkyl (e.g., methyl, or ethyl); and

each m is, independently, 1, 2, 3, or 4;

wherein each aryl or heteroaryl is, independently, optionally substituted with up to five R⁴ selected from the group consisting of halogen, hydroxyl, cyano, amino, (C₁₋₆ alkyl)amino, di(C₁₋₆ alkyl)amino, C₁₋₆ alkyl, C₁₋₆ haloalkyl, C₃₋₆ cycloalkyl, C₁₋₆ alkoxy, C₁₋₆ haloalkoxy, C₃₋₆ cycloalkoxy, arylalkyl (e.g., benzyl) and heteroaryl (e.g., pyridine, furan, thiophene, indole);

with the provisos that

(i) when each R¹ is ethyl, or one R¹ is H and the other R¹ is Na [i.e., the moiety —P(═O)(OR¹)(OR¹) is represented —P(═O)(OH)(O⁻Na⁺)], then R₂ is not

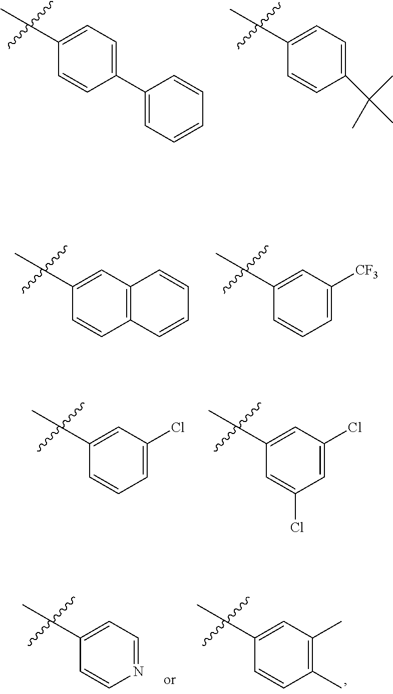

where the squiggly line ( [symbol] ) represents the point of attachment of R₂ to the reset of the molecule, and

(ii) when each R₁ is ethyl, or each R₁ is NH₄, then R₂ is not unsubstituted phenyl.

In another aspect, the present invention relates to a pharmaceutical composition comprising a compound as disclosed in any embodiment herein and a pharmaceutically acceptable excipient.

In another aspect, the present invention relates to a herbicide comprising a compound as disclosed in any embodiment herein and a pharmaceutically acceptable excipient.

In another aspect, the present invention relates to a method for treating or preventing a microbial infection in a subject (e.g., a subject in need thereof) comprising administering to the subject an effective amount of a compound as disclosed in any embodiment herein. In some embodiments, the microbial infection is malaria. In some embodiments, the microbial infection is tuberculosis.

In another aspect, the present invention relates to a method for treating or preventing a pathogen in a subject (e.g., a subject in need thereof) comprising administering to the subject an effective amount of a compound as disclosed in any embodiment herein. In some embodiments, the pathogen is Plasmodium falciparum or related Plasmodium parasite species, Mycobacteriun tuberculosis or related Mycobacterium bacteria species, Saureus, or ESK APE pathogen.

### BRIEF DESCRIPTION OF THE DRAWINGS

FIG. 1 depicts the methyl erythritol phosphate (MEP) pathway of isoprenoid biosynthesis.

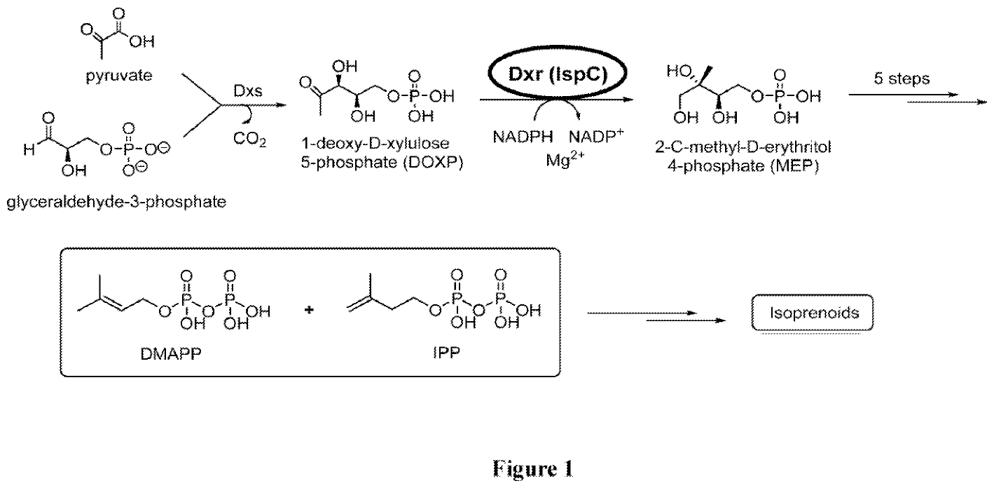

### DETAILED DESCRIPTION OF THE INVENTION

As used herein the following definitions shall apply unless otherwise indicated.

The term “in need thereof” refers to a subject infected with a microbial pathogen or at risk of becoming infected by the microbial pathogen. In some cases, the microbial pathogen is a eukaryotic pathogen, and more specifically a eukaryotic pathogen belonging to the genus Plasmodium. In some cases the pathogen is a prokaryotic pathogen, and more specifically belonging to the genus Mycobacterium.

As used throughout, the phrase an “effective amount” of a compound of this disclosure is measured by the therapeutic effectiveness of the compound, wherein at least one adverse effect of a disorder is ameliorated or alleviated. More specifically, administering a compound or composition results in complete or at least partial inhibition of a metabolic pathway or other biological processes in a pathogen. In addition, an effective amount is sufficient to result in at least some degree of alleviation or prevention of an infection caused by a pathogen, or prevention of an infection by the pathogen.

The terms “treating or preventing” are intended to include preventing, eradicating, or inhibiting the resulting increase of undesired physiological activity associated with a disorder or infection, for example, in the context of the therapeutic or prophylactic methods of the invention. In another embodiment, the term treating or preventing includes antagonistic effects, e.g., diminishment of the activity or production of mediators of a disorder.

As used herein and unless otherwise indicated, the term “formulation” refers to a composition comprising a compound of the present disclosure that is described in a particular dosage form (e.g., tablet) or with a particular dosage amount.

When administered to a subject (e.g., to an animal for veterinary use or to a human for clinical use), the compounds of the invention can be optionally administered in isolated form.

The phrase “pharmaceutically acceptable salt(s),” as used herein includes but is not limited to salts of acidic or basic groups that may be present in compounds of the present disclosure. Compounds in the present disclosure that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds in the present disclosure that include an amino moiety may form pharmaceutically acceptable salts with various amino acids, in addition to the acids mentioned above. Compounds in the present disclosure that are acidic in nature are capable of forming base salts with various pharmacologically acceptable cations. Examples of such salts include alkali metal or alkaline earth metal salts and ammonium salts, for example, calcium, magnesium, sodium, potassium, lithium, zinc, potassium, and iron salts.

As used herein and unless otherwise indicated, the terms “prodrug” or “pharmaceutically acceptable prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise oligonucleotides, peptides, lipids, aliphatic and aromatic groups, or NO, NO₂, ONO, and ONO₂ moieties. Prodrugs can typically be prepared using well known methods, such as those described in Burger's Medicinal Chemistry and Drug Discovery, pp. 172, 178, 949, 982 (Manfred E. Wolff ed., 5th ed. 1995), and Design of Prodrugs (H. Bundgaard ed., Elselvier, New York 1985).

The phrase “pharmaceutically acceptable excipient” may be any substance, not itself a therapeutic agent, used as a carrier, diluent, adjuvant, binder, and/or vehicle for delivery of a therapeutic agent to a patient, or added to a pharmaceutical composition to improve its handling or storage properties or to permit or facilitate formation of a compound or pharmaceutical composition into a unit dosage form for administration. Pharmaceutically acceptable excipients are known in the pharmaceutical arts and are disclosed, for example, in Remington: The Science and Practice of Pharmacy, 21st Ed. (Lippincott Williams & Wilkins, Baltimore, M D, 2005). As will be known to those in the art, pharmaceutically acceptable excipients can provide a variety of functions and can be described as wetting agents, buffering agents, suspending agents, lubricating agents, emulsifiers, disintegrants, absorbents, preservatives, surfactants, colorants, flavorants, and sweeteners.

In the present disclosure, the term “halo” or “halogen” as used by itself or as part of another group refers to —Cl, —F, —Br, or —I. In one embodiment, the halo is —Cl or —F. In one embodiment, the halo is —Cl.

In the present disclosure, the term “nitro” as used by itself or as part of another group refers to —NO₂.

In the present disclosure, the term “cyano” as used by itself or as part of another group refers to —CN.

In the present disclosure, the terms “hydroxy” and “hydroxyl” as used by itself or as part of another group refers to —OH.

In the present disclosure, the term “alkyl” as used by itself or as part of another group refers to unsubstituted straight- or branched-chain aliphatic hydrocarbons containing from one to twelve carbon atoms, i.e., C₁₋₁₂ alkyl, or the number of carbon atoms designated, e.g., a C1 alkyl such as methyl, a C2 alkyl such as ethyl, a C3 alkyl such as propyl or isopropyl, a C₁₋₃ alkyl such as methyl, ethyl, propyl, or isopropyl, and so on. In one embodiment, the alkyl is a C₁₋₁₀ alkyl. In another embodiment, the alkyl is a C₁₋₆ alkyl. In another embodiment, the alkyl is a C₁₋₄ alkyl. In another embodiment, the alkyl is a straight chain C₁₋₁₀ alkyl. In another embodiment, the alkyl is a branched chain C₃₋₁₀ alkyl. In another embodiment, the alkyl is a straight chain C₁₋₆ alkyl. In another embodiment, the alkyl is a branched chain C₃₋₆ alkyl. In another embodiment, the alkyl is a straight chain C₁₋₄ alkyl. In another embodiment, the alkyl is a branched chain C₃₋₄ alkyl. In another embodiment, the alkyl is a straight or branched chain C₃₋₄ alkyl. Non-limiting exemplary C₁₋₁₀ alkyl groups include methyl, ethyl, propyl, isopropyl, butyl, sec-butyl, tert-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tert-butyl, and iso-butyl.

In the present disclosure, the term “cycloalkyl” as used by itself or as part of another group refers to saturated and partially unsaturated (containing one or two double bonds) cyclic aliphatic hydrocarbons containing one to three rings having from three to twelve carbon atoms, i.e., C₃₋₁₂ cycloalkyl. or the number of carbons designated. In one embodiment, the cycloalkyl group has two rings. In one embodiment, the cycloalkyl group has one ring. In another embodiment, the cycloalkyl group is chosen from a C₃₋₈ cycloalkyl group. In another embodiment, the cycloalkyl group is chosen from a C₃₋₆ cycloalkyl group. Non-limiting exemplary cycloalkyl groups include cyclopropyl, cyclobutyl, cyclopentyl, cyclohexyl, cycloheptyl, cyclooctyl, norbornyl, decalin, adamantyl, cyclohexenyl, and cyclopentenyl, cyclohexenyl.

In the present disclosure, the term “alkenyl” as used by itself or as part of another group refers to an alkyl group as defined above containing one, two or three carbon-to-carbon double bonds. In one embodiment, the alkenyl group is chosen from a C₂₋₆ alkenyl group. In another embodiment, the alkenyl group is chosen from a C₂₋₄ alkenyl group. Non-limiting exemplary alkenyl groups include ethenyl, propenyl, isopropenyl, butenyl, see-butenyl, pentenyl, and hexenyl.

In the present disclosure, the term “alkynyl” as used by itself or as part of another group refers to an alkyl group as defined above containing one to three carbon-to-carbon triple bonds. In one embodiment, the alkynyl has one carbon-to-carbon triple bond. In one embodiment, the alkynyl group is chosen from a C₂₋₆ alkynyl group. In another embodiment, the alkynyl group is chosen from a C₂₋₄ alkynyl group. Non-limiting exemplary alkynyl groups include ethynyl, propynyl, butynyl, 2-butynyl, pentynyl, and hexynyl groups.

In the present disclosure, the term “haloalkyl” as used by itself or as part of another group refers to an alkyl group substituted by one or more fluorine, chlorine, bromine and/or iodine atoms. In one embodiment, the alkyl group is substituted by one, two, or three fluorine and/or chlorine atoms. In another embodiment, the haloalkyl group is a C₁₋₆ haloalkyl group In another embodiment, the haloalkyl group is a C₁₋₄ haloalkyl group. Non-limiting exemplary haloalkyl groups include fluoromethyl, 2-fluoroethyl, difluoromethyl, trifluoromethyl, pentafluoroethyl, 1, 1-difluoroethyl, 2,2-difluoroethyl, 2,2,2-trifluoroethyl, 3,3,3-trifluoropropyl, 4,4,4-trifluorobutyl, and trichloromethyl groups.

In the present disclosure, the term “alkoxy” as used by itself or as part of another group refers to an optionally substituted alkyl, optionally substituted cycloalkyl, optionally substituted alkenyl or optionally substituted alkynyl attached to a terminal oxygen atom. In one embodiment, the alkoxy group is chosen from a C₁₋₄ alkoxy group. In another embodiment, the alkoxy group is chosen from a C₁₋₆ alkoxy group. In another embodiment, the alkoxy group is chosen from a C₁₋₄ alkyl attached to a terminal oxygen atom, e.g., methoxy, ethoxy, and tert-butoxy.

In the present disclosure, the term “haloalkoxy” as used by itself or as part of another group refers to a C₁₋₄ haloalkyl attached to a terminal oxygen atom. Non-limiting exemplary haloalkoxy groups include fluoromethoxy, difluoromethoxy, trifluoromethoxy, and 2,2,2-trifluoroethoxy.

In the present disclosure, the term “aryl” as used by itself or as part of another group refers to a monocyclic, bi cyclic, or tricyclic aromatic ring system having from six to fourteen carbon atoms, i.e., C₆-C₁₄ aryl. Non-limiting exemplary aryl groups include phenyl (abbreviated as “Ph”), 1-naphthyl, 2-naphthyl, phenanthryl, anthracyl, indenyl, azulenyl, biphenyl, biphenylenyl, and fluorenyl groups. In one embodiment, the aryl group is chosen from phenyl, 1-naphthyl, or 2-naphthyl. In one embodiment, the aryl is a bicyclic or tricyclic C₁₀-C₁₋₄ aromatic ring system.

The term “heteroaryl”, unless otherwise specified, refers to an optionally substituted 5-to-14-member aromatic ring having one or more heteroatoms selected from N, O, and S as ring atoms. The heteroaryl may be a mono-, bi- or tricyclic ring system. Examples of such “heterocyclic ring” or “heteroaryl” radicals include, but are not limited to, oxazolyl, thiazolyl, imidazolyl, pyrrolyl, furanyl, pyridinyl, pyrimidinyl, pyrazinyl, benzofuranyl, indolyl, benzothiazolyl, benzoxazolyl, carbazolyl, quinolyl, isoquinolyl, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, 4-piperidonyl, pyrrolidinyl, pyridazinyl, oxazolinyl, oxazolidinyl, triazolyl, indanyl, isoxazolyl, isoxazolidinyl, morpholinyl, thiazolinyl, thiazolidinyl, isothiazolyl, quinuclidinyl, isothiazolidinyl, isoindolyl, indolinyl, isoindolinyl, octahydroindolyl, octahydroisoindolyl, decahydroisoquinolyl, benzimidazolyl, thiadiazolyl, benzopyranyl, tetrahydrofuryl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, and isochromanyl. The heteroaryl ring radical may be attached to the main structure at any heteroatom or carbon atom. The term “substituted heteroaryl” also includes ring systems substituted with one or more oxide (═O) substituents, such as pyridinyl N-oxides.

The term “arylalkyl”, unless otherwise specified, refers to an aryl group as defined above directly bonded to an alkyl group as defined above, e.g., —CH₂C₆H₅ and —C₂H₅C₆H₅.

In the present disclosure, the term “optionally substituted aryl” as used herein by itself or as part of another group means that the aryl as defined above is either unsubstituted or substituted with one to five substituents independently selected from the group consisting of halogen, hydroxy, nitro, cyano, —SCH₃, —SCF₃, —NR₁₀R₁₁, —C(═O)NR₁₀R₁₁, —C(═O)R₁₃, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₁₂ cycloalkyl, optionally substituted C₆-C₁₄ aryl, optionally substituted 5— to 14-membered heteroaryl, and optionally substituted 3— to 14-membered heterocyclic ring, wherein R₁₀ and R₁ are independently selected from the group consisting of hydrogen and C₁₋₆ alkyl; or R₁₀ and R₁ taken together with the nitrogen atom to which they are attached form a 3— to 12-membered heterocyclic ring and R₁₃ is C₁₋₄ alkyl.

In one embodiment, the optionally substituted aryl is an optionally substituted phenyl. In one embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 4-bromophenyl, 4-triflurophenyl, 2,6-di-fluoropheny 1, 2, 6-di-chlorophenyl, 2-methyl, 3-methoxyphenyl, 2-ethyl, 3-methoxyphenyl, 3,4-di-methoxyphenyl, 3,5-di-fluorophenyl, 3,4-di-chlorophenyl, 3,5-di-methylphenyl, 3,5-dimethoxy, 4-methylphenyl, 2-fluoro-3-chlorophenyl, 3-chloro-4-fluorophenyl.

Additional non-limiting exemplary substituted aryl groups include 4-isopropylphenyl, 4-chlorophenyl, 4-bromophenyl, 4-fluorophenyl, 4-methoxyphenyl, 4-methylphenyl, and 4-trifluoromethylphenyl.

The term optionally substituted aryl is also meant to include groups having fused optionally substituted cycloalkyl and fused optionally substituted heterocyclic rings. Non-limiting examples include:

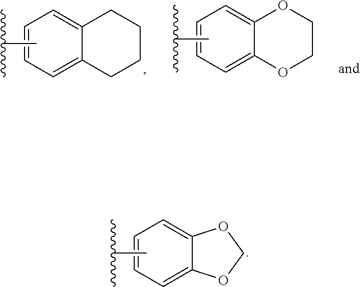

Certain of the compounds described herein may contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. Non-limiting examples of intermediate mixtures include a mixture of isomers in a ratio of 10:90, 13:87, 17:83, 20:80, or 22:78. Optically active (R)- and (S)-isomers can be prepared using chiral synthons or chiral reagents or resolved using conventional techniques. When the compounds described herein contain olefinic double bonds or other centers of geometric asymmetry, and unless specified otherwise, it is intended that the compounds include both E and Z geometric isomers.

The terms “tautomer” and “tautomers” refer to compounds which are characterized by relatively easy interconversion of isomeric forms in equilibrium. These isomers are intended to be covered by this invention. “Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g. in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

Additionally, the present invention also includes the compounds which differ only in the presence of one or more isotopically enriched atoms for example replacement of hydrogen with deuterium or tritium, or the replacement of a carbon by ¹³C- or ¹⁴C-enriched carbon.

The compounds of the present invention may also contain unnatural proportions of atomic isotopes at one or more of atoms that constitute such compounds. For example, the compounds may be radio-labelled with radioactive isotopes, such as for example tritium (3H), iodine-125 (¹²⁵I) or carbon-14 (¹⁴C). All isotopic variations of the compounds of the present invention, whether radioactive or not, are encompassed within the scope of the present invention.

The term “comprising” (and related terms such as “comprise,” “comprises,” “having” or “including”) includes those embodiments, for example, an embodiment of any composition of matter, composition, method, or process, or the like, that “consist of” or “consist essentially of” the described features.

The term “subject” or “patient” refers to an animal, such as a mammal, for example a human. The methods and uses described herein can be useful in both human therapeutics and veterinary applications (e.g., dogs, cats, cows, sheep, pigs, horses, goats, chickens, turkeys, ducks, and geese).

In some embodiments, the subject is a mammal, and in some embodiments, the subject is a human.

The term “pharmaceutically acceptable excipient” includes, but is not limited to, any and all solvents, dispersion media, coatings, antibacterial and antifungal agents, isotonic and absorption delaying agents, one or more suitable diluents, fillers, salts, disintegrants, binders, lubricants, glidants, wetting agents, controlled release matrices, colorants/flavoring, carriers, buffers, stabilizers, solubilizers, and combinations thereof. Except insofar as any conventional media or agent is incompatible with the active ingredient, its use in the therapeutic compositions of the invention is contemplated. Supplementary active ingredients can also be incorporated into the compositions.

Dxr inhibitors are described in, for example, U.S. Pat. No. 9,593,136 and International Publication Nos. WO 19/005982 and WO 17/12780, each of which are incorporated herein by reference in their entirety.

The present invention provides a series of compounds as antimicrobial agents that work via DXR inhibition.

In one aspect, the present invention relates to a compound of formula (I)

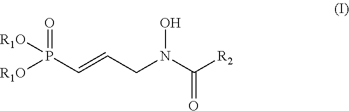

or a tautomer thereof, stereoisomer thereof, prodrug thereof, or pharmaceutically acceptable salt thereof,

wherein

each R₁ is, independently, H, C₁₋₆ alkyl, C₁₋₆ haloalkyl, C₃₋₆ cycloalkyl, —(CR^{a}R^{b})ₘ-aryl, —(CR^{a}R^{b})ₘ—O(C═O)—C₁₋₆ alkyl (e.g., —CH₂O—C(═O)—C(CH₃)₃), —(CR^{a}R^{b})—O(C═O)—C₃₋₆ cycloalkyl, —(CR^{a}R^{b})ₘ—O(C═O)-aryl (e.g., —CH₂—O—C(═O)—C₆H₅), —(CR^{a}R^{b})—O(C═O)O—C₁₋₆ alkyl, or —(CR^{a}R^{b})ₘ—O(C═O)O—C₃₋₆ cycloalkyl, —NH₄, —N(alkyl)₄ or —N(aryl)₄, wherein the atom at the left is attached to the oxygen atom;

R² is aryl (e.g., phenyl, naphthyl) or heteroaryl (e.g., pyridinyl);

each of R¹ and R^{b} is, independently, H, halogen, or C₁₋₄ alkyl (e.g., methyl, or ethyl); and

each in is, independently, 1, 2, 3, or 4;

wherein each aryl or heteroaryl is, independently, optionally substituted with up to five R⁴ selected from the group consisting of halogen, hydroxyl, cyano, amino, (C₁₋₆ alkyl)amino, di(C₁₋₆ alkyl)amino, C₁₋₆ alkyl, C₁₋₆ haloalkyl, C₃₋₆ cycloalkyl, C₁₋₆ alkoxy, C₁₋₆ haloalkoxy, C₃₋₆ cycloalkoxy, arylalkyl (e.g., benzyl) and heteroaryl (e.g., pyridine, furan, thiophene, indole);

with the provisos that

(i) when each R¹ is ethyl, or one R¹ is H and the other R¹ is Na [i.e., the moiety —P(═O)(OR¹)(OR¹) is represented —P(═O)(OH)(O⁻Na⁺)], then R₂ is not

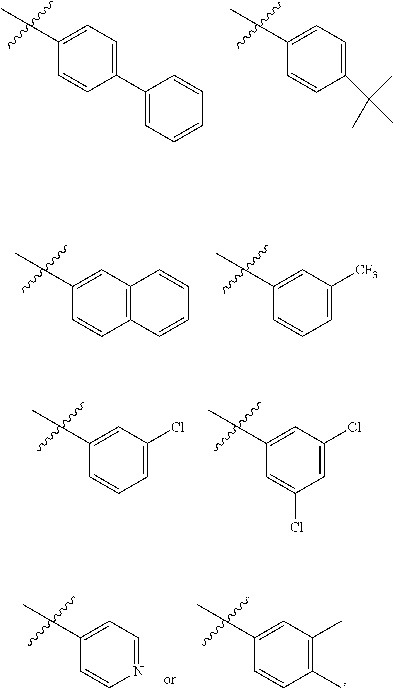

where the squiggly line ( [symbol] ) represents the point of attachment of R₂ to the reset of the molecule, and

[symbol]

(ii) when each R₁ is ethyl, or each R₁ is NH₄, then R₂ is not unsubstituted phenyl.

In one aspect, the present invention relates to a compound of formula (I)

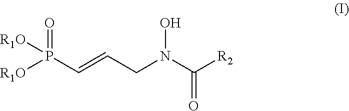

or a tautomer thereof, stereoisomer thereof, prodrug thereof, or pharmaceutically acceptable salt thereof,

wherein

each R₁ is, independently, H, C₁₋₆ alkyl, C₁₋₆ haloalkyl, C₃₋₆ cycloalkyl, —(CR^{a}R^{b})ₘ-aryl —(CR^{a}R^{b})ₘ—O(C═O)—C₁₋₆ alkyl (e.g., —CH₂—O—C(O)—C(CH₃)₃), —(CR^{a}R^{b})—O(C═O)—C₃₋₆ cycloalkyl, —(CR^{a}R^{b})ₘ—O(C═O)-aryl (e.g., —CH₂—O—C(═O)—C₆H₅), —(CR^{a}R^{b})ₘ—O(C═O)O—C₁₋₆ alkyl, or —(CR^{a}R^{b})ₘ—O(C═O)O—C₃₋₆ cycloalkyl, —NH₄, —N(alkyl), or —N(aryl)₄, wherein the atom at the left is attached to the oxygen atom;

R₂ is aryl (e.g., phenyl, naphthyl) or heteroaryl (e.g., pyridinyl);

each of R^{a} and R^{b} is, independently, H, halogen, or C₁₋₄ alkyl (e.g., methyl, or ethyl);

each m is, independently, 1, 2, 3, or 4;

wherein each aryl or heteroaryl is, independently, optionally substituted with up to five R⁴ selected from the group consisting of halogen, hydroxyl, cyano, amino, (C₁₋₆ alkyl)amino, di(C₁₋₆ alkyl)amino, C t alkyl, C t haloalkyl, C₃₋₆ cycloalkyl, C₁₋₆ alkoxy, C₁₋₆ haloalkoxy, C₃₋₆ cycloalkoxy, arylalkyl (e.g., benzyl) and heteroaryl (e.g., pyridine, furan, thiophene, indole);

with the provisos that

(i) when each R₁ is ethyl, or one R₁ is H and the other R₁ is Na [i.e., the moiety —P(═O)(OR¹)(OR¹) is represented —P(═O)(OH)(O⁻Na⁺)], then R₂ is not

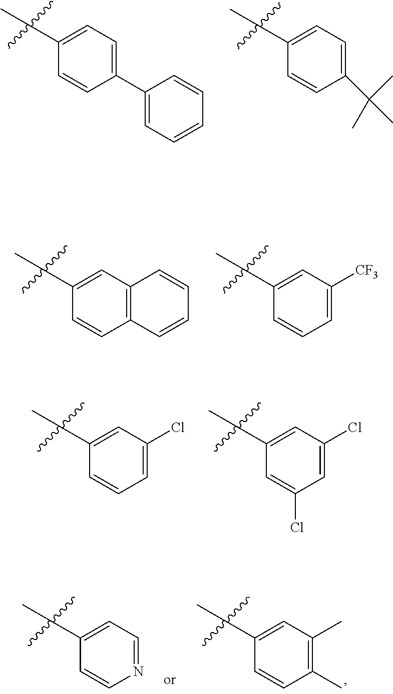

where the squiggly line ( [symbol] ) represents the point of attachment of R₂ to the reset of the molecule, and
(ii) when each R₁ is ethyl, or each R₁ is NH₄, then R₂ is not unsubstituted phenyl; and
(iii) when each R₁ is POM, then R₂ is not unsubstituted phenyl, 4-flurophenyl, 3-trifluromethylphenyl, 3-chlorophenyl or 3,4-dichlorophenyl.

In some embodiments of any of the compounds of formula (I), the compound is a mono-salt. In some embodiments of any of the compounds of formula (I), the compound is a di-salt.

In some embodiments of any of the compounds of formula (I) the salt is a quaternary ammonium salt. In some of any of the compounds of formula (I), the salt is a NH₄ salt. In some embodiments of any of the compounds of formula (I), the salt is a di-quaternary ammonium salt. In some embodiments of any of the compounds of formula (I), the salt is a di-NH₄ salt.

In some embodiments of any of the compounds of formula (I), each R₁ is N₁₋₄.

In some embodiments of any of the compounds of formula (I), each R₁ is C₁₋₄ alkyl.

In some embodiments of any of the compounds of formula (I), each R₁ is ethyl.

In some embodiments of any of the compounds of formula (I), each R₁ is —(CR^{a}R^{b})ₘ—O(C═O)—C₁₋₆ alkyl. In some embodiments of any of the compounds of formula (I), each R₁ is —CH₂O(C═O)—C₁₋₆ alkyl. In some embodiments of any of the compounds of formula (I), each R₁ is —CH(CH₃)—O(C═O)—C₁₋₆ alkyl. In some embodiments of any of the compounds of formula (I), each R₁ is —CH₂—O(C═O)—C(CH₃)₃ (pivaloyloxymethyl, POM).

In some embodiments of any of the compounds of formula (I), each R₁ is —(CR^{a}R^{b})ₘ—O(C═O)-aryl. In some embodiments of any of the compounds of formula (I), each R₁ is —(CR^{a}R^{b})ₘ—O(C═O)-phenyl. In some embodiments of any of the compounds of formula (I), each R₁ is —CH₂—O(C═O)-aryl. In some embodiments of any of the compounds of formula (I), each R₁ is —CH₂—O(C═O)-phenyl (benzyloxymethyl, BOM).

In some embodiments of any of the compounds of formula (I), m is 1, 2, or 3. In some embodiments of any of the compounds of formula (I), m is 1 or 2. In some embodiments of any of the compounds of formula (I), m is 1.

In some embodiments of any of the compounds of formula (I), R₂ is selected from phenyl, 4-thiomethylphenyl, 3-flurophenyl, 4-fluorophenyl, 4-methylphenyl, 4-trifluoromethylphenyl, 1-naphthyl, 4-chlorophenyl, 4-methoxyphenyl, 3-chlorophenyl, 2-naphthyl, 4-methoxyphenyl, 4-t-butylphenyl, 3-trifluoromethylphenyl, 4-pyridinyl, 3,5-dichlorophenyl, 3,4-dichlorophenyl, and 4-biphenyl.

Examples of compounds of the present invention include, but are not limited to, those shown in Table 1 below, and tautomers, stereoisomers, prodrugs and pharmaceutically acceptable salt thereof. If only one R¹ group is listed in Table 1, both R¹ groups are the same.

TABLE 1

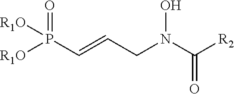

| Compound # | R¹ | R² |
|---|---|---|
| 1 | Na/H | phenyl |
| 2 | Na/H | 4-thiomethylphenyl |
| 3 | Na/H | 3-flurophenyl |
| 4 | Na/H | 4-fluorophenyl |
| 5 | Na/H | 4-methylphenyl |
| 6 | Na/H | 4-trifluoromethylphenyl |
| 7 | Na/H | 1-naphthyl |
| 8 | Na/H | 4-chlorophenyl |
| 9 | Na/H | 4-methoxyphenyl |
| 10 | Et | 4-thiomethylphenyl |
| 11 | Et | 3-flurophenyl |
| 12 | Et | 4-fluorophenyl |
| 13 | Et | 4-methylphenyl |
| 14 | Et | 4-trifluoromethylphenyl |
| 15 | Et | 1-naphthyl |
| 16 | Et | 4-chlorophenyl |
| 17 | Et | 3-chlorophenyl |
| 18 | Et | 2-naphthyl |
| 19 | Et | 4-methoxyphenyl |
| 20 | POM | phenyl |
| 21 | POM | 4-thiomethylphenyl |
| 22 | POM | 3-flurophenyl |
| 23 | POM | 4-fluorophenyl |
| 24 | POM | 4-methylphenyl |
| 25 | POM | 4-trifluoromethylphenyl |
| 26 | POM | 1-naphthyl |
| 27 | POM | 4-chlorophenyl |
| 28 | POM | 3-chlorophenyl |
| 29 | POM | 2-naphthyl |
| 30 | POM | 4-t-butylphenyl |
| 31 | POM | 3-trifluoromethylphenyl |
| 32 | POM | 4-pyridinyl |
| 33 | POM | 3,5-dichlorophenyl |
| 34 | POM | 3,4-dichlorophenyl |
| 35 | POM | 4-methoxyphenyl |
| 36 | POM | 4-biphenyl |
| 37 | BOM | phenyl |
| 38 | BOM | 4-thiomethylphenyl |
| 39 | BOM | 3-flurophenyl |
| 40 | BOM | 4-fluorophenyl |
| 41 | BOM | 4-methylphenyl |
| 42 | BOM | 4-trifluoromethylphenyl |
| 43 | BOM | 1-naphthyl |
| 44 | BOM | 4-chlorophenyl |
| 45 | BOM | 3-chlorophenyl |
| 46 | BOM | 2-naphthyl |
| 47 | BOM | 4-t-butylphenyl |
| 48 | BOM | 3-trifluoromethylphenyl |
| 49 | BOM | 4-pyridinyl |
| 50 | BOM | 3,5-dichlorophenyl |
| 51 | BOM | 3,4-dichlorophenyl |
| 52 | BOM | 4-methoxyphenyl |
| 53 | BOM | 4-biphenyl |
| 54 | NH₄ | 4-thiomethylphenyl |
| 55 | NH₄ | 3-flurophenyl |
| 56 | NH₄ | 4-fluorophenyl |
| 57 | NH₄ | 4-methylphenyl |
| 58 | NH₄ | 4-trifluoromethylphenyl |
| 59 | NH₄ | 1-naphthyl |
| 60 | NH₄ | 4-chlorophenyl |
| 61 | NH₄ | 3-chlorophenyl |
| 62 | NH₄ | 2-naphthyl |
| 63 | NH₄ | 4-t-butylphenyl |
| 64 | NH₄ | 3-trifluoromethylphenyl |
| 65 | NH₄ | 4-pyridinyl |
| 66 | NH₄ | 3,5-dichlorophenyl |
| 67 | NH₄ | 3,4-dichlorophenyl |
| 68 | NH₄ | 4-methoxyphenyl |

### Pharmaceutical Compositions

The present invention provides a pharmaceutical composition comprising one or more compounds of the present invention, or a pharmaceutically acceptable salt thereof.

The pharmaceutical compositions described herein may include one or more additional active ingredients as described herein. The pharmaceutical composition may be administered for any of the disorders described herein.

The pharmaceutical compositions described herein are typically formulated to provide a therapeutically effective amount of a compound of the present invention as the active ingredient. Where desired, the pharmaceutical compositions contain a compound of the present invention as the active ingredient and one or more pharmaceutically acceptable carriers or excipients, such as inert solid diluents and fillers, diluents, including sterile aqueous solution and various organic solvents, permeation enhancers, solubilizers and adjuvants.

The pharmaceutical compositions can be administered alone or in combination with one or more other agents, which are also typically administered in the form of pharmaceutical compositions. Where desired, the subject compounds and other agent(s) may be mixed into a preparation or both components may be formulated into separate preparations to use them in combination separately or at the same time.

Methods and uses described herein include administration of a compound of the present invention by itself, or in combination as described herein, and in each case optionally including one or more suitable diluents, fillers, salts, disintegrants, binders, lubricants, glidants, wetting agents, controlled release matrices, colorants/flavorings, carriers, excipients, buffers, stabilizers, solubilizers, and combinations thereof.

Preparations of various pharmaceutical compositions are well known in the art., see, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999), all of which are incorporated by reference herein in their entirety.

The compounds or pharmaceutical compositions of the present invention can be administered by any route that enables delivery of the compound(s) to their intended site of action, such as oral routes, intraduodenal routes, parenteral injection (including intravenous, intraarterial, subcutaneous, intramuscular, intravascular, intraperitoneal or infusion), topical administration (e.g. transdermal application), rectal administration, via local delivery by catheter or stent or through inhalation. The compounds can also be administered intraadiposally or intrathecally.

The compositions described herein can be administered in solid, semi-solid, liquid or gaseous form, or may be in dried powder, such as lyophilized form. The pharmaceutical compositions can be packaged in forms convenient for delivery, including, for example, solid dosage forms such as capsules, sachets, cachets, gelatins, papers, tablets, capsules, suppositories, pellets, pills, troches, and lozenges. The type of packaging will generally depend on the desired route of administration. Implantable sustained release formulations are also contemplated, as are transdermal formulations.

### Synthesis

The following general methodology described in the schemes below provides the manner and process of making and using the compounds of the present invention and are illustrative rather than limiting. Further modification of the provided methodology may also be devised to achieve and serve the purpose of the invention. Accordingly, there may be other embodiments which fall within the spirit and scope of the invention as defined by this specification.

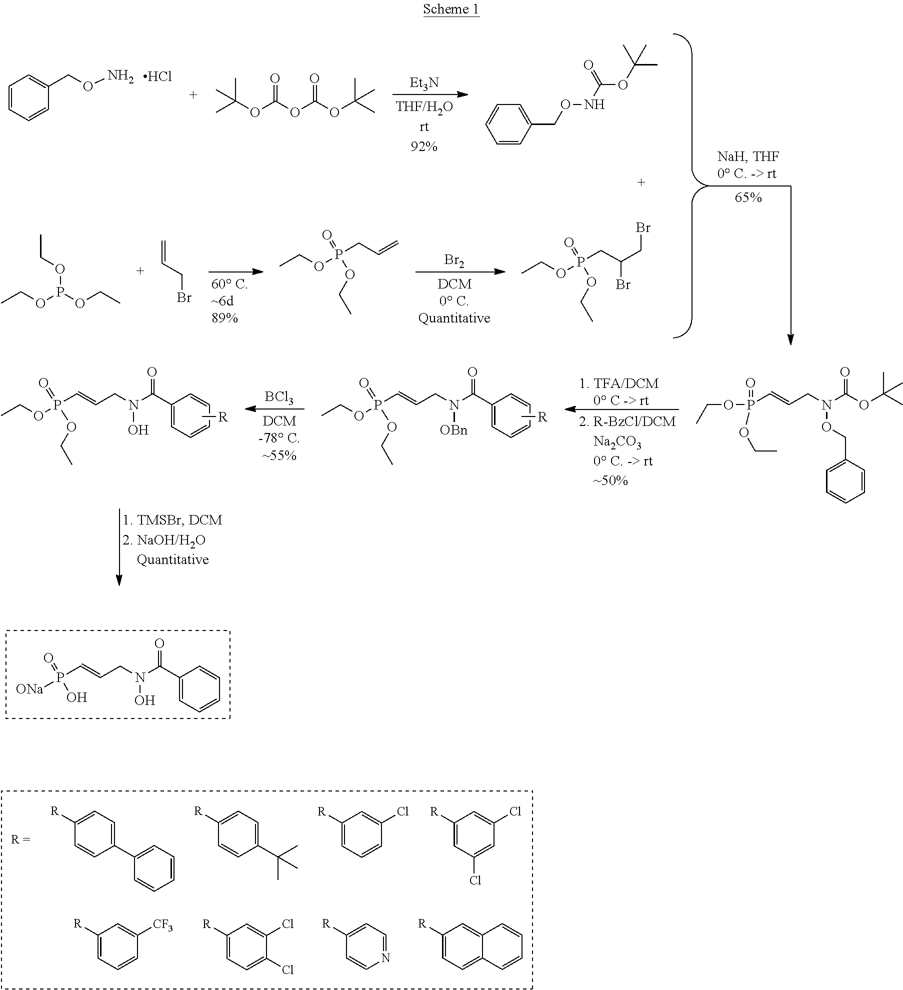

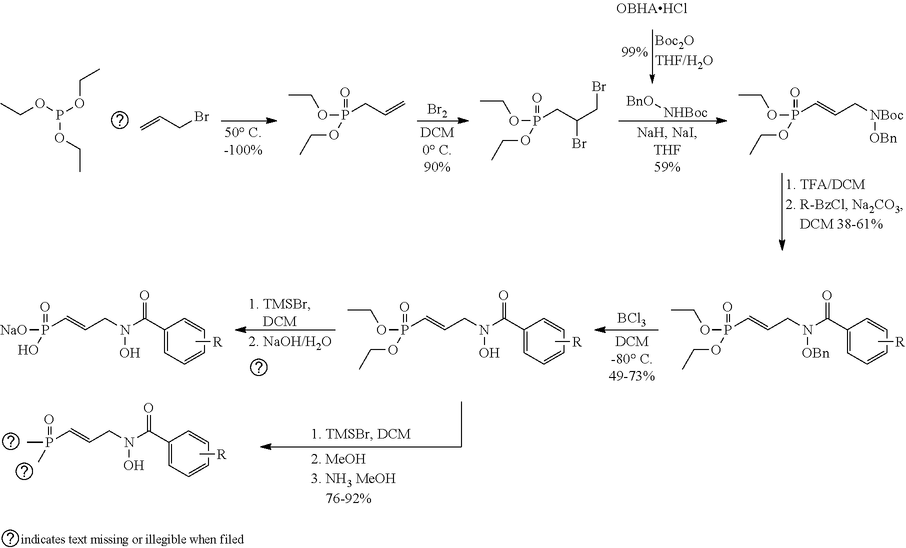

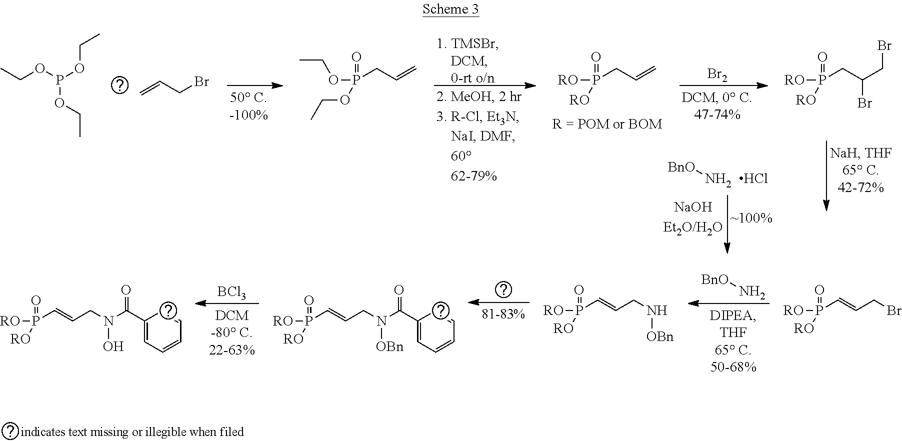

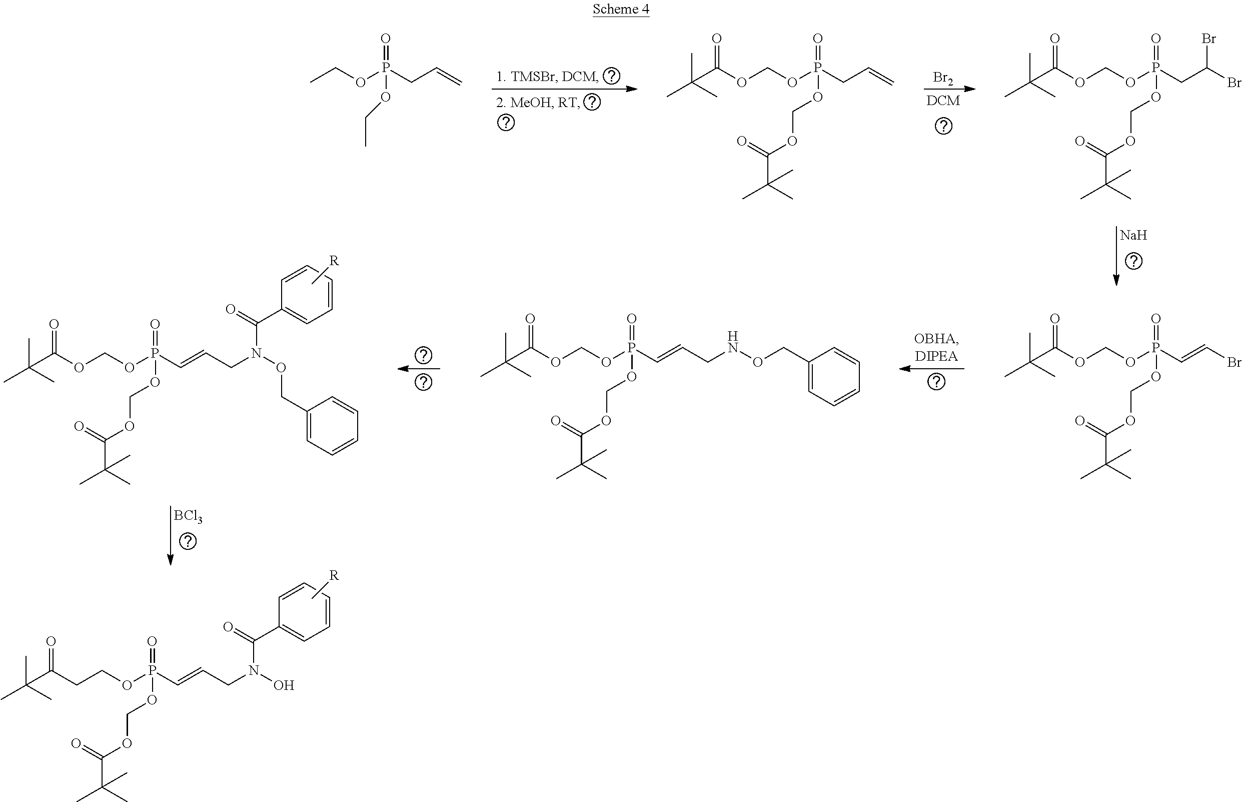

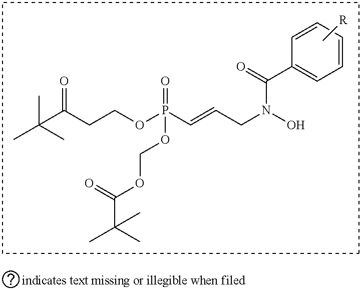

### Plasmodium Falciparum IC₅₀ Data

Some Plasmodium Falciparum DXR and whole cell IC₅₀ data is provided in Table 2 below

TABLE 2

| Compound | | | P. f. DXR | P. f. |
|---|---|---|---|---|
| # | R₁ | R₂ | IC₅₀ (μm) | IC₅₀ (μm) |
| | Na/H | H | 0.092 | 0.019 |
| | Na/H | CH₃ | 0.206 | 0.202 |
| | NH₄ | phenyl | 0.081 | 0.273 |
| | NH₄ | 4-fluorophenyl | 0.038 | 0.173 |
| | Na/H | 4-fluorophenyl | 0.041 | 0.121 |
| | Na/H | 4-methylphenyl | 0.164 | 1.389 |
| | Na/H | 4-trifluoromethylphenyl | 0.146 | 2.499 |
| | Na/H | 1-naphthyl | 1.797 | 5.479 |
| | Na/H | 4-chlorophenyl | 0.44 | 0.317 |
| | Na/H | 3-chlorophenyl | 0.158 | 0.279 |
| | Na/H | 4-biphenyl | 0.290 | 4.378 |
| | Na/H | 2-naphthyl | 0.471 | 1.625 |
| | Na/H | 4-t-butylphenyl | (37.9)^{a} | 43.706 |
| | Na/H | 3-trifluoromethylphenyl | 0.048 | 0.188 |
| | Na/H | 4-pyridinyl | 0.519 | 6.525 |
| | Na/H | 3,5-dichlorophenyl | 0.064 | 0.170 |
| | NH₄ | 3-chlorophenyl | | 0.116 |
| | NH₄ | 4-chlorophenyl | | 0.301 |
| | NH₄ | 3,5-dichlorophenyl | | 0.218 |
| | NH₄ | 3-trifluoromethylphenyl | 0.049 | 0.106 |
| | NH₄ | 3-trifluoromethylphenyl | | 4.777 |
| | NH₄ | 3,4-dichlorophenyl | | 0.129 |
| | NH₄ | 4-methoxyphenyl | | 0.632 |
| | NH₄ | 3-fluorophenyl | | 0.352 |
| | NH₄ | 4-fluorophenyl | | 0.173 |
| | NH₄ | 4-pyridinyl | | 5.388 |
| | POM | H | | 0.013 |
| | POM | CH₃ | | 0.018 |
| | POM | phenyl | | 0.200 |
| | BOM | H | | 0.042 |
| | BOM | CH₃ | | 0.011 |
| | BOM | phenyl | | 0.297 |

### EXAMPLES

### Diethyl (prop-2-en-1-yl)phosphonate

This compound was synthesized as previously reported (1).

### Diethyl (2,3-dibromopropyl)phosphonate

This compound was synthesized as previously reported (1).

### Tert-butyl N-(benzyloxy)carbamate

This compound was synthesized as previously reported (1).

### Tert-butyl N-(benzyloxy)—N-[(2E)-3-(Diethoxyphosphoryl)prop-2-en-1-yl]carbamate

This compound was synthesized as previously reported (1).

(1) Jackson et al., Bioorganic & Medicinal Chemistry Letters, 24(2), 649-653, 2014.

### Diethyl [(1E)-3-[N-(benzyloxy)-1-phenylformamido]prop-1-en-1-yl]phosphonate

To a solution of tert-butyl N-(benzyloxy)—N-[(2E)-3-(Diethoxyphosphoryl) prop-2-en-1-yl]carbamate (1 eq) in CH₂Cl₂ (0.6 M) at 0° C., trifluoroacetic acid (0.3 M) was added dropwise. The reaction was stirred overnight while allowed to warm to ambient temperature. The reaction was quenched with saturated NaHCO₃and extracted with CH₂Cl₂, washed with brine, dried with Na₂SO₄, and concentrated in vacuo. The crude concentrate was redissolved in CH₂Cl₂ (0.3 M) and Na₂CO₃ (5 eq) was added. Aroyl chlorides (2 eq) were added dropwise to the reaction mixture at 0° C., which was allowed to warm to ambient temperature and stir overnight. The reaction was quenched with saturated NaHCO₃and extracted with CH₂Cl₂, washed with brine, dried with Na₂SO₄, and concentrated in vacuo. Chromatographic separation of the crude concentrate on silica gel (dichloromethane/ethyl acetate) afforded the title compound. Light yellow oil (0.70 g, 36%). ¹H NMR (400 MHz, CDCl₃) δ 7.59 (d, J=7.6 Hz, 2H), 7.45-7.26 (m, 3H), 7.18 (q, J=6.5 Hz, 3H), 7.00 (d, J=6.3 Hz, 2H), 6.76 (tt, J=17.2, 5.1 Hz, 1H), 6.04-5.81 (m, 1H), 4.61 (s, 2H), 4.56-4.40 (m, 2H), 3.99 (p, J=7.3 Hz, 4H), 1.21 (t, J=7.1 Hz, 6H). LCMS (ESI+): 426 m/z [M+Na]+

### Diethyl [(1E)-3-(N-hydroxy-1-phenylformamido)prop-1-en-1-yl]phosphonate

Upon dissolution of diethyl [(1E)-3-[N-(benzyloxy)-1-phenylformamido]prop-1-en-1-yl]phosphonate (1.0 eq) in CH₂Cl₂ (0.3 M), the solution was cooled to −78° C. BCl₃ (1.0M in dichloromethane, 5 eq) was added dropwise and the resulting mixture was stirred at −78° C. until completion of the reaction when monitored by thin layer chromatography (TLC).

Upon quenching with cold sodium bicarbonate, the mixture was extracted with dichloromethane, washed with brine, and dried with Na₂SO₄. After concentration in vacuo, the crude mixture was purified via column chromatography (silica gel, dichloromethane/ethyl acetate) to afford the hydroxamate. Yellow oil (0.44 g, 82%). ¹H NMR (400 MHz, CDCl₃) δ 9.81 (s, 1H), 7.49 (s, 2H), 7.32-7.08 (m, 3H), 6.71-6.50 (m, 1H), 5.87-5.67 (m, 1H), 4.28 (s, 2H), 3.76 (p, J=7.2 Hz, 4H), 1.08-1.03 (m, 6H)

### ({[(1E)-3-[N-(benzyloxy)-1-phenylformamido]prop-1-en-1-yl]({[(2,2-dimethylpropanoyl)oxy]methoxy})phosphoryl}oxy)methyl 2,2-dimethylpropanoate

To a solution of diethyl [(1E)-3-[N-(benzyloxy)-1-phenylformamido]prop-1-en-1-yl]phosphonate (1 eq) in dry CH₂Cl₂ (0.1 M) under N₂ was added TMSBr (10 eq) dropwise at 0° C. The reaction mixture was warmed to room temperature and stirred overnight, concentrated. The mixture was dissolved in CH₂Cl₂, evaporated and dried under vacuum. The crude was then stirred in 0.5 M NaOH (2 eq) in H₂O at room temperature for 1 h, washed with Et₂O (3×) and lyophilized to give disodium salts as white solids. The crude solids was then dissolved in dry DMF (0.1 M), added TEA (6 eq), chloromethyl pivalate (6 eq) and NaI (0.1 eq). The reaction mixture was stirred at 60° C. for 24 h, quenched with H₂O, extracted with Et₂O (3×). The combined organic layers were dried with anhydrous Na₂SO₄, filtered and concentrated. The crude was then purified by column chromatography on silica gel using Hexanes and EtOAc or CH₂Cl₂ and EtOAc to give the pure title compound. Light yellow oil (182 mg, 32%). ¹H NMR (400 MHz, CDCl₃) δ 7.74-7.01 (m, 1OH), 6.95-6.77 (m, 1H), 6.08-5.93 (m, 1H), 5.75-5.58 (m, 4H), 4.65 (s, 2H), 4.51-4.46 (m, 2H), 1.20 (s, 18H). ¹³C NMR (101 MHz, CDCl₃) δ 176.7, 170.1, 147.2 (d, J=6.0 Hz), 133.6, 133.5, 130.9, 129.4, 128.9, 128.4, 128.3, 128.0, 118.6 (d, J=192.6 Hz), 81.5 (d, J=5.4 Hz), 77.0, 49.5 (d, J=25.4 Hz), 38.6, 26.7. LC-MS (ESI+): 576.2 m/z [M+H]⁺.

### (E)-(((3-(N-hydroxybenzamido)allyl)phosphoryl)bis(oxy))bis(methylene)bis(2,2-dimethylpropanoate)

To a solution of ({[(1E)-3-[N-(benzyloxy)-1-phenylformamido]prop-1-en-1-yl]({[(2,2-dimethylpropanoyl)oxy]methoxy})phosphoryl}oxy)methyl 2,2-dimethylpropanoate (1 eq) in dry CH₂Cl₂ (0.1 M) under N₂ was added boron trichloride (1M in CH₂Cl₂, 4 eq) at −78° C. dropwise. The reaction mixture was stirred at −78° C. for 30 min to 3 h, quenched with saturated NaHCO₃(aq) and extracted with EtOAc (5×). The combined organic layers were dried with anhydrous Na₂SO₄, filtered and concentrated. The crude was then purified by column chromatography on silica gel using EtOAc and MeOH (EtOAc and CH₂Cl₂ to give the pure title compound. Light yellow oil (27 mg, 30%). ¹H NMR (400 MHz, CDCl₃) δ 8.69 (s, 1H), 7.57-7.34 (m, 5H), 6.82 (ddt, J=21.8, 17.2, 4.5 Hz, 1H), 6.04 (ddt, J=20.6, 17.2, 1.7 Hz, 1H), 5.67-5.60 (m, 4H), 4.45-4.38 (m, 2H), 1.19 (s, 18H). ¹³C NMR (101 MHz, CDCl₃) δ 176.9, 167.7, 146.8, 131.3, 128.7, 128.5, 128.0, 118.7 (d, J=192.4 Hz), 81.5 (d, J=5.2 Hz), 52.7 (d, J=24.3 Hz), 38.7, 26.8. LC-MS (ESI*): 486.2 m/z [M+H]⁺, 971.2 m/z [2M+H]⁺. HRMS (FAB⁺) calculated for C₂₂H₃₂NO₉P, 485.1815; found, 486.1877 [M+H]⁺.

### Sodium hydrogen [(1E)-3-[1-(3,5-dichlorophenyl)—N-hydroxyformamido]prop-1-en-1-yl]phosphonate

Upon dissolution of diethyl [(1E)-3-[1-(3,5-dichlorophenyl)—N-hydroxyformamido]prop-1-en-1-yl]phosphonate (1 eq) in CH₂Cl₂ (0.3 M), the solution was cooled to 0° C. Bromotrimethylsilane (4 eq) was added dropwise, and the reaction was allowed to warm to room temperature and stirred overnight. The mixture was concentrated in vacuo and re-dissolved in water (0.1 M) and aqueous sodium hydroxide (1.05 eq) was added. After stirring for two hours, the mixture was frozen and subsequently lyophilized to afford the mono-sodium salt of the phosphonic acid. Yellow gum (0.10 g, quant). ¹H NMR (400 MHz, DMSO) δ 7.77 (d, J=2.0 Hz, 1H), 7.69 (s, 1H), 7.62 (s, 1H), 6.39-6.23 (m, 1H), 5.87 (t, J=16.4 Hz, 1H), 4.27 (s, 2H). LCMS (ESI−): 324, 326 m/z [M−H]⁻.

### Diammonium [(1E)-3-(N-hydroxy-1-phenylformamido)prop-1-en-1-yl]phosphonate

Upon dissolution of diethyl [(1E)-3-(N-hydroxy-1-phenylformamido)prop-1-en-1-yl]phosphonate (1 eq) in CH₂Cl₂ (0.25M), the solution was cooled to 0° C. Bromotrimethylsilane (4 eq) was added dropwise, and the reaction was allowed to warm to room temperature and stirred overnight. The mixture was concentrated in vacuo and re-dissolved in methanol (0.25M). After stirring for two hours, the mixture was concentrated in vacuo and re-dissolved in a solution of 7N NH₃ in methanol (2.0 eq) and stirred for an hour. The mixture was evacuated in vacuo to afford the di-ammonium salt of the phosphonic acid. Tan solid (0.30 g, 75%). ¹H NMR (400 MHz, CD30D) 6 7.38 (s, 2H), 7.18 (dq, J=14.5, 6.9 Hz, 3H), 6.27 (s, 1H), 5.82 (s, 1H), 4.15 (s, 2H). LCMS (ESI+): 258 m/z [M-(2xNH3)+H]⁺. HRMS (ESI+) calc'd for C₁₀H₁₃NO₅P: 258.0531; found 258.0526 [M+H]⁺

### ({[(Benzoyloxy)methoxy](prop-2-en-1-yl)phosphoryl}oxy)methyl benzoate

Diethyl (prop-2-en-1-yl)phosphonate (6.8 g, 37.9 mmol) was dissolved in dichloromethane (150 mL). The solution was cooled to 0° C. and bromotrimethylsilane (20.0 mL, 152 mmol) was added slowly. The reaction was allowed to warm to ambient temperature, stir overnight, and was then concentrated in vacuo. The crude residue was then dissolved and stirred in methanol (100 mL) for two hours, followed by concentration in vacuo. The crude mixture at this point was dissolved in THE (70 mL), followed by addition of chloromethyl benzoate (15.5 g, 152 mmol), triethylamine (21.3 mL, 152 mmol), and sodium iodide (0.57 g, 3.8 mmol). The mixture was stirred at 65° C. overnight. The reaction was then quenched with water, extracted with dichloromethane, washed with brine, dried with Na₂SO₄, and concentrated in vacuo. Purification via column chromatography (silica gel, dichloromethane/ethyl acetate) afforded the title compound as a yellow oil (11.8 g, 79%). ¹H NMR (400 MHz, CDCl₃) δ 8.04 (ddt, J=7.8, 1.3, 0.6 Hz, 4H), 7.66-7.55 (m, 2H), 7.49-7.38 (m, 4H), 5.97-5.83 (m, 4H), 5.82-5.63 (m, 1H), 5.25-5.06 (m, 2H), 2.82-2.66 (m, 2H). ¹³C NMR (101 MHz, CDCl₃) δ 164.89, 133.83, 130.00, 128.69, 128.56, 125.69, 125.57, 121.44, 121.29, 81.86, 81.80, 32.94, 31.55. LCMS (ESI+): 391 m/z [M+H]({[(Benzoyloxy)methoxy](2,3-dibromopropyl)phosphoryl}oxy)methyl benzoate To a solution of ({[(Benzoyloxy)methoxy](prop-2-en-1-yl)phosphoryl}oxy)methyl benzoate (2.4 g, 6.13 mmol) in dichloromethane (40 mL), Br2 (0.38 mL, 7.36 mmol) was added dropwise at 0° C. Upon completion of the reaction by monitoring via thin-layer chromatography, the reaction was quenched with saturated sodium bisulfite, extracted with dichloromethane, washed with brine, and dried with Na₂SO₄. After concentration in vacuo, the crude mixture was purified via column chromatography (silica gel, hexanes/ethyl acetate) to afford the title compound as a yellow oil (2.5 g, 74%). ¹H NMR (400 MHz, CDCl₃) δ 8.12-8.00 (m, 4H), 7.59 (ddd, J=8.5, 6.7, 1.2 Hz, 2H), 7.50-7.37 (m, 4H), 6.04-5.89 (m, 4H), 4.50-4.35 (m, 1H), 3.88 (ddd, J=10.8, 4.4, 3.0 Hz, 1H), 3.70 (dd, J=10.7, 8.0 Hz, 1H), 3.05-2.87 (m, 1H), 2.62-2.44 (m, 1H). ¹³C NMR (101 MHz, CDCl₃) δ 164.91, 164.87, 133.95, 130.07, 128.61, 128.52, 128.49, 81.99, 81.93, 81.90, 81.84, 42.02, 41.98, 37.41, 37.27, 35.22, 33.79. LCMS (ESI+): 549, 551, 553 m/z [M+H]⁺. [({[(E)-benzoyloxy]methoxy}[(1E)-3-bromoprop-1-en-1-yl]phosphoryl)oxy]methyl benzoate To a stirring solution of ({[(Benzoyloxy)methoxy](2,3-dibromopropyl)phosphoryl}oxy)methyl benzoate (2.5 g, 4.53 mmol) in THF (30 mL), sodium hydride in mineral oil (60%, 0.22 g, 5.43 mmol) was added. The reaction was heated to 65° C. overnight. Upon cooling, the reaction was quenched with sodium bicarbonate, extracted with ethyl acetate, washed with brine, and dried with Na₂SO₄. After concentration in vacuo, the crude mixture was purified via column chromatography (silica gel, hexanes/ethyl acetate) to afford the title compound as a colorless gum (1.27 g, 60%). ¹H NMR (400 MHz, CDCl₃) δ 8.04 (dt,J=8.3, 1.5 Hz, 4H), 7.63-7.53 (m, 2H), 7.49-7.41 (m, 4H), 6.98-6.77 (m, 1H), 6.05-5.92 (m, 5H), 3.85 (dd, J=6.5, 1.5 Hz, 2H). ¹³C NMR (101 MHz, CDCl₃) δ 164.77, 147.19, 147.11, 133.87, 130.06, 128.59, 128.57, 121.15, 119.24, 81.98, 81.92, 30.05, 29.76. LCMS (ESI+): 469, 471 m/z [M+H]⁺

### [({[(E)-benzoyloxy]methoxy}[(1E)-3-[(benzyloxy)amino]prop-1-en-1-yl]phosphoryl)oxy]methyl benzoate

To a stirring solution of [({[(E)-benzoyloxy]methoxy}[(1E)-3-bromoprop-1-en-1-yl]phosphoryl)oxy]methyl benzoate (6.8 g, 14.5 mmol) in THE (100 mL), 8 (2.14 g, 17.4 mmol) and diisopropylethylamine (4.8 mL, 29.0 mmol) were added. The reaction was heated to 65° C. overnight. Upon cooling, the reaction was quenched with water, extracted with ethyl 204 acetate, washed with brine, and dried with Na₂SO₄. After concentration in vacuo, the crude mixture was purified via column chromatography (silica gel, hexanes/ethyl acetate) to afford the title compound as a yellow oil (3.7 g, 50%). With recovery of unreacted allylic bromide (0.87 g), the corrected yield is 58%. ¹H NMR (400 MHz, CDCl₃) δ 8.07-7.95 (m, 4H), 7.58-7.48 (m, 2H), 7.43-7.33 (m, 4H), 7.32-7.17 (m, 5H), 6.97-6.79 (m, 1H), 6.03-5.85 (m, 5H), 5.54 (s, 1H), 4.56 (s, 2H), 3.49 (ddd, J=5.1, 3.0, 1.8 Hz, 2H). ¹³C NMR (101 MHz, CDCl₃) δ 164.75, 150.71, 150.66, 137.56, 133.77, 130.00, 130.00, 128.71, 128.57, 128.53, 128.52, 128.40, 128.36, 127.87, 118.48, 116.57, 81.92, 81.87, 53.97, 53.73. LCMS (ESI+): 512 m/z [M+H]⁺

### [({[(E)-benzoyloxy]methoxy}[(1E)-3-[N-(benzyloxy)-1-phenylformamido]prop-1-en-1-yl]phosphoryl)oxy]methyl benzoate

To a stirring solution of [({[(E)-benzoyloxy]methoxy}[(1E)-3-[(benzyloxy)amino]prop-1-en-1-yl]phosphoryl)oxy]methyl benzoate (0.1.0 g, 1.98 mmol) in dichloromethane (20 mL), benzoyl chloride (0.28 mL, 2.37 mmol) and diisopropylethylamine (0.68 mL, 3.96 mmol) were added. After 2 hours, the reaction was quenched with sodium bicarbonate, extracted with dichloromethane, washed with brine, and dried with Na₂SO₄. After concentration in vacuo, the crude mixture was purified via column chromatography (silica gel, hexanes/ethyl acetate) to afford the title compound as a light-yellow oil (1.0 g, 83%). ¹H NMR (400 MHz, CDCl₃) δ 8.00 (d, J=7.8 Hz, 4H), 7.66-7.50 (m, 4H), 7.46-7.33 (m, 7H), 7.22 (dt, J=13.9, 6.6 Hz, 3H), 7.04-6.83 (m, 3H), 6.10-5.89 (m, 5H), 4.56 (s, 2H), 4.46-4.32 (m, 2H). ¹³C NMR (101 MHz, CDCl₃) δ 170.18, 164.80, 147.65, 147.60, 133.85, 133.79, 133.73, 133.58, 130.88, 130.02, 129.44, 128.93, 128.63, 128.58, 128.55, 128.49, 128.38, 128.34, 128.07, 119.57, 117.65, 81.99, 81.93, 49.91, 49.66. LCMS (ESI+): 616 m/z [M+H]⁺

### [({[(E)-benzoyloxy]methoxy}[(1E)-3-(N-hydroxy-1-phenylformamido)prop-1-en-1-yl]phosphoryl)oxy]methyl benzoate

Upon dissolution of [({[(E)-benzoyloxy]methoxy}[(1E)-3-[N-(benzyloxy)—1-phenylformamido]prop-1-en-1-yl]phosphoryl)oxy]methyl benzoate (0.15 g, 0.244 mmol) in dichloromethane (0.5 mL), the solution was cooled to −78° C. BCl₃ (1.0M in dichloromethane, 1.2 mL) was added dropwise and the resulting mixture was stirred at −78° C. for 3 hours. Upon quenching with cold sodium bicarbonate, the mixture was extracted with dichloromethane, washed with brine, and dried with Na₂SO₄. After concentration in vacuo, the crude mixture was purified via column chromatography (silica gel, dichloromethane/ethyl acetate) to afford the title compound as a yellow oil (0.046 g, 35%). ¹H NMR (400 MHz, CDCl₃) δ 8.00 (dd, J=8.1, 3.9 Hz, 4H), 206 7.54 (qd, J=9.9, 4.7 Hz, 4H), 7.39 (dt, J=11.9, 5.7 Hz, 5H), 7.31 (dt, J=11.4, 5.5 Hz, 2H), 6.97-6.79 (m, 1H), 6.19-6.03 (m, 1H), 5.89 (tt, J=13.7, 5.0 Hz, 4H), 4.33 (s, 2H). ¹³C NMR (101 MHz, CDCl₃) δ 164.86, 147.45, 133.88, 131.10, 130.02, 129.98, 129.77, 128.57, 128.50, 128.37, 127.99, 127.39, 119.35, 117.44, 82.00, 81.95, 52.73. LCMS (ESI+): 526 m/z [M+H]⁺. HRMS (ESI+) calc'd for C₂₆H₂₅NO₉P: 526.1267; found 526.1267 [M+H]⁺

### Chloromethyl Benzoate

To a stirring solution of ZrCl₄ (29.8 g, 128 mmol) in dichloromethane (200 mL) was added benzoyl chloride (20.0 g, 142 mmol). The solution was stirred for 15 minutes and then cooled to 0° C. A solution of trioxane (4.7 g, 52.5 mmol) in dichloromethane (11 mL) was added slowly and the resulting mixture was allowed to warm to ambient temperature. After 3 hours, the mixture was cooled to 0° C. and quenched with water. The mixture was extracted with dichloromethane, washed with brine, dried with Na₂SO₄, and concentrated in vacuo. After crystallization of side products in hexanes, concentration of the filtrate afforded the title compound as a yellow oil (20.8 g, 96%). ¹H NMR (400 MHz, CDCl₃) δ 8.08 (d, J=8.5 Hz, 2H), 7.61 (ddd, J=8.6, 6.6, 1.3 Hz, 1H), 7.52-7.44 (m, 2H), 5.96 (s, 2H)

### Bacterial Strains and Growth Conditions

Recombinant protein was expressed in Escherichia coli Rosetta2(DE3) cells obtained from Novagen (San Diego, CA). E. coli was cultured at 37° C. in Luria-Bertani (LB) media supplemented with 100 μg/mL ampicillin and 34 μg/ml chloramphenicol with constant shaking at 250 rpm. Agar (1.5% wt/vol) was added to prepare solid media.

Cloning. Expression, and Purification of P falciparum DXR

The P falciparum dxr gene was truncated to begin at Lys 75 to remove the apicoplast signaling sequence. A Pf 3D7 trophozoite cDNA library (MRA-297) was acquired from BEi resources and used as the template for amplification of the PIDXR gene. The gene was PCR amplified using primers 5′ CACC AAG AAA CCA ATT AAT GTA GCA 3′ forward and 5′ CTA TAG AGA ATT ATG TTT GTT GTA TAT ATC GGT AG 3′ reverse and cloned into a pETl 00/D-TOPO vector to yield pPIDXR, facilitating the expression of an N-terminal His6-tagged protein.

The expression plasmid (pPIDXR) was separately transformed into chemically competent E. coli Rosetta2(DE3) cells for protein expression. To express the His-tagged protein, a 10 mL overnight seed culture was added to IL of LB media and then incubated with shaking at 37° C. and 250 rpm. At an OD600 of 1.8, protein expression was induced with addition of isopropyl b-D-thiogalactopyranoside (IPTG) to 0.5 mM and the culture was further incubated with shaking at 37° C. and 250 rpm for an additional 18 hours. Cells were harvested via centrifugation (4648×g, 20 min, 4° C.) and stored at −80° C. Protein was subsequently isolated and purified from the cells via chemical lysis and affinity chromatography.

Cells were lysed with lysis buffer A (100 mM Tris pH 8.0, 0.032% lysozyme, 3 mL per gram cell pellet), followed by lysis buffer B (0.1 M CaCh, 0.1 M MgCh, 0.1 M NaCl, 0.020% DNase, 0.3 mL per gram cell pellet). Clarified cell lysate was collected after centrifugation (48,000×g, 20 min, 4° C.) and passed through a TALON immobilized metal affinity column (Clontech Laboratories, Mountain View, CA).

The column was washed with 20 column volumes of Ix equilibrium buffer (50 mM HEPES pH 7.5, 300 mM NaCl), 10 column volumes of Ix wash buffer (50 mM HEPES pH 7.5, 300 mM NaCl, 10 mM imidazole), and 15 column volumes of 2× wash buffer (100 mM HEPES pH 7.5, 600 mM NaCl, 20 mM imidazole). The protein was eluted with 5 column volumes of Ix elution buffer (150 mM imidazole pH 7.0, 300 mM NaCl). Buffer was exchanged with 0.1 M Tris pH 7.5, I mM NaCl, 5 mM DTT during concentration by ultrafiltration. Protein concentration was determined using Advanced Protein Assay Reagent (Cytoskeleton, Denver CO) with y-globulins (Sigma-Aldrich) as the standard. Purified protein was visualized via Coomassie stained SDS-PAGE. The yield of PIDXR averages I mg per IL shake flask.

P falciparum Culture

P falciparum strain 3D7 (wild-type, WT) was obtained through MR4 as part of the BEi Resources Repository, NIAID, NIH (www.mr4.org). AP falciparum strain containing increased levels of MEP pathway metabolites, had]

(MRA-1257), and its isogenic compliment, had]+ Pfl-Iadl-GFP (MRA-1258), were generated in strain 3D7, as reported (Guggisberg et al.). Parasites were cultured in a 2% suspension of human erythrocytes and RPMI 1640 (Sigma) medium supplemented with 27 mM sodium bicarbonate, 11 mM glucose, 5 mM HEPES, I mM sodium pyruvate, 0.37 mM hypoxanthine, 0.01 mM thymidine, 10 μg/mL gentamicin, and 0.5% Albumax (Gibco) at 37° C., 5% 02/5% CO₂/90% N₂ atmosphere.

The description of the present embodiments of the invention has been presented for purposes of illustration, but is not intended to be exhaustive or to limit the invention to the form disclosed. Many modifications and variations will be apparent to those of ordinary skill in the art. As such, while the present invention has been disclosed in connection with an embodiment thereof, it should be understood that other embodiments may fall within the spirit and scope of the invention. Patents and publications cited herein are incorporated by reference in their entirety.

1. A compound of formula (I) 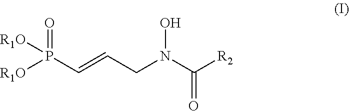
or a tautomer thereof, stereoisomer thereof, prodrug thereof, or pharmaceutically acceptable salt thereof,
wherein
each R₁ is, independently, H, C₁₋₆ alkyl, C₁₋₆ haloalkyl, C₃₋₆ cycloalkyl, —(CR^{a}R^{b})ₘ-aryl, —(CR^{a}R^{b})ₘ—O(C═O)—C₁₋₆ alkyl (e.g., —CH₂—O—C(═O)—C(CH₃)₃), —(CR^{a}R^{b})ₘ—O(C═O)—C₃₋₆ cycloalkyl, —(CR^{a}R^{b})ₘ—O(C═O)-aryl (e.g., —CH₂—O—C(═O)—C₆H₅), —(CR^{a}R^{b})ₘ—O(C═O)O—C₁₋₆ alkyl, or —(CR^{a}R^{b})ₘ—O(C═O)O—C₃₋₆ cycloalkyl, —NH₄, —N(alkyl)₄ or —N(aryl)₄, wherein the atom at the left is attached to the oxygen atom;
R² is aryl (e.g., phenyl, naphthyl) or heteroaryl (e.g., pyridinyl);
each of R^{a} and R^{b} is, independently, H, halogen, or C₁₋₄ alkyl (e.g., methyl, or ethyl); and
each m is, independently, 1, 2, 3, or 4;
wherein each aryl or heteroaryl is, independently, optionally substituted with up to five R⁴ selected from the group consisting of halogen, hydroxyl, cyano, amino, (C₁₋₆ alkyl)amino, di(C₁₋₆ alkyl)amino, C₁₋₆ alkyl, C₁₋₆ haloalkyl, C₃₋₆ cycloalkyl, C₁₋₆ alkoxy, C₁₋₆ haloalkoxy, C₃₋₆ cycloalkoxy, arylalkyl (e.g., benzyl) and heteroaryl (e.g., pyridine, furan, thiophene, indole);
with the provisos that
(i) when each R¹ is ethyl, or one R¹ is H and the other R¹ is Na [i.e., the moiety —P(═O)(OR¹)(OR¹) is represented —P(═O)(OH)(O⁻Na⁺)], then R₂ is not 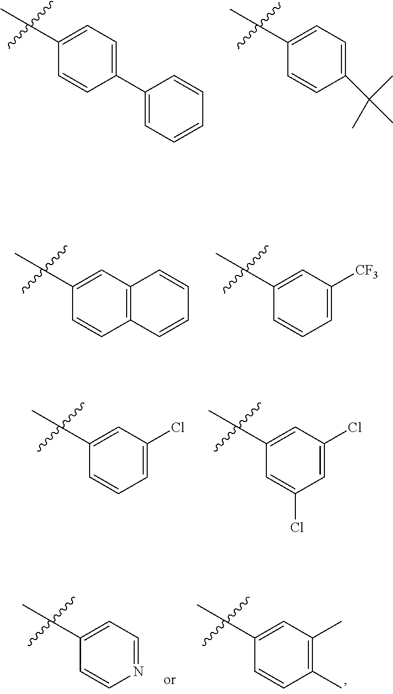
where the squiggly line ( [symbol] ) represents the point of attachment of R₂ to the reset of the molecule, and [symbol]
(ii) when each R₁ is ethyl, or each R₁ is NH₄, then R₂ is not unsubstituted phenyl.

2. A compound of formula (I) 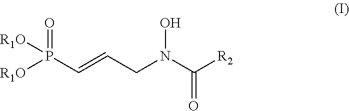
or a tautomer thereof, stereoisomer thereof, prodrug thereof, or pharmaceutically acceptable salt thereof,
wherein
each R₁ is, independently, H, C₁₋₆ alkyl, C₁₋₆ haloalkyl, C₃₋₆ cycloalkyl, —(CR^{a}R^{b})ₘ-aryl, —(CR^{a}R^{b})ₘ—O(C═O)—C₁₋₆ alkyl (e.g., —CH₂—O—C(═O)—C(CH₃)₃), —(CR^{a}R^{b})ₘ—O(C═O)—C₃₋₆ cycloalkyl, —(CR^{a}R^{b})ₘ—O(C═O)-aryl (e.g., —CH₂—O—C(═O)—C₆H₅), —(CR^{a}R^{b})ₘ—O(C═O)O—C₁₋₆ alkyl, or —(CR^{a}R^{b})ₘ—O(C═O)O—C₃₋₆ cycloalkyl, —NH₄, —N(alkyl)₄ or —N(aryl)₄, wherein the atom at the left is attached to the oxygen atom;
R₂ is aryl (e.g., phenyl, naphthyl) or heteroaryl (e.g., pyridinyl);
each of R^{a} and R^{b} is, independently, H, halogen, or C₁₋₄ alkyl (e.g., methyl, or ethyl);
each m is, independently, 1, 2, 3, or 4;
wherein each aryl or heteroaryl is, independently, optionally substituted with up to five R⁴ selected from the group consisting of halogen, hydroxyl, cyano, amino, (C₁₋₆ alkyl)amino, di(C₁₋₆ alkyl)amino, C₁₋₆ alkyl, C₁₋₆ haloalkyl, C₃₋₆ cycloalkyl, C₁₋₆ alkoxy, C₁₋₆ haloalkoxy, C₃₋₆ cycloalkoxy, arylalkyl (e.g., benzyl) and heteroaryl (e.g., pyridine, furan, thiophene, indole);
with the provisos that
(i) when each R¹ is ethyl, or one R₁ is H and the other R₁ is Na [i.e., the moiety —P(═O)(OR¹)(OR¹) is represented —P(═O)(OH)(O⁻Na⁺)], then R₂ is not 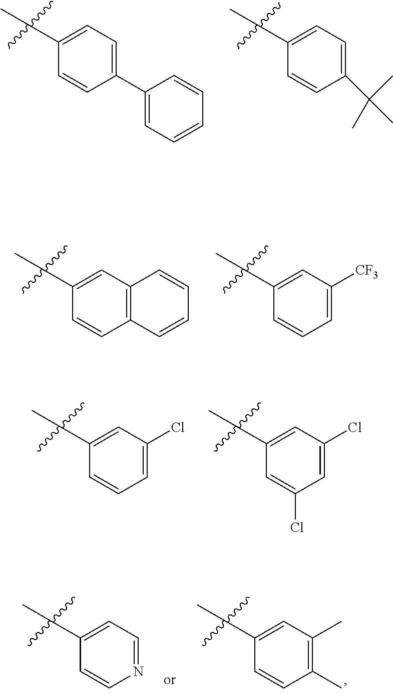
where the squiggly line ( [symbol] ) represents the point of attachment of R₂ to the reset of the molecule, and [symbol]
(ii) when each R₁ is ethyl, or each R₁ is NH₄, then R₂ is not unsubstituted phenyl; and
(iii) when each R₁ is POM, then R₂ is not unsubstituted phenyl, 4-flurophenyl, 3-trifluromethylphenyl, 3-chlorophenyl or 3,4-dichlorophenyl.

3. The compound according to claim 1, wherein the compound is a mono-salt.

4. The compound according to claim 1, wherein the compound is a di-salt.

5. The compound according to claim 1, wherein the compound is a di-NH₄ salt.

6. The compound according to claim 1, wherein each R₁ is NH₄.

7. The compound according to claim 1, wherein each R₁ is C₁₋₄ alkyl.

8. The compound according to claim 1, wherein each R₁ is ethyl.

9. The compound according to claim 1, wherein each R₁ is —(CR^{a}R^{b})ₘ—O(C═O)—C₁₋₆ alkyl.

10. The compound according to claim 1, wherein each R₁ is —CH₂O(C═O)—C₁₋₆ alkyl.

11. The compound according to claim 1, wherein each R₁ is —CH₂—O(C═O)—C(CH₃)₃ (pivaloyloxymethyl, POM).

12. The compound according to claim 1, wherein each R₁ is —(CR^{a}R^{b})ₘ—O(C═O)-aryl.

13. The compound according to claim 1, wherein each R₁ is —(CR^{a}R^{b})ₘ—O(C═O)—C₆H₅.

14. The compound according to claim 1, wherein m is 1, 2, or 3.

15. The compound according to claim 1, wherein m is 1 or 2.

16. The compound according to claim 1, wherein R₂ is selected from phenyl, 4-thiomethylphenyl, 3-flurophenyl, 4-fluorophenyl, 4-methylphenyl, 4-trifluoromethylphenyl, 1-naphthyl, 4-chlorophenyl, 4-methoxyphenyl, 3-chlorophenyl, 2-naphthyl, 4-methoxyphenyl, 4-t-butylphenyl, 3-trifluoromethylphenyl, 4-pyridinyl, 3,5-dichlorophenyl, 3,4-dichlorophenyl, and 4-biphenyl.

17. A compound according to claim 1, wherein the compound is selected from:

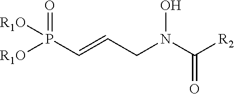

| Compound # | R¹ | R² |
|---|---|---|
| 1 | Na/H | phenyl |
| 2 | Na/H | 4-thiomethylphenyl |
| 3 | Na/H | 3-flurophenyl |
| 4 | Na/H | 4-fluorophenyl |
| 5 | Na/H | 4-methylphenyl |
| 6 | Na/H | 4-trifluoromethylphenyl |
| 7 | Na/H | 1-naphthyl |
| 8 | Na/H | 4-chlorophenyl |
| 9 | Na/H | 4-methoxyphenyl |
| 10 | Et | 4-thiomethylphenyl |
| 11 | Et | 3-flurophenyl |
| 12 | Et | 4-fluorophenyl |
| 13 | Et | 4-methylphenyl |
| 14 | Et | 4-trifluoromethylphenyl |
| 15 | Et | 1-naphthyl |
| 16 | Et | 4-chlorophenyl |
| 17 | Et | 3-chlorophenyl |
| 18 | Et | 2-naphthyl |
| 19 | Et | 4-methoxyphenyl |
| 20 | POM | phenyl |
| 21 | POM | 4-thiomethylphenyl |
| 22 | POM | 3-flurophenyl |
| 23 | POM | 4-fluorophenyl |
| 24 | POM | 4-methylphenyl |
| 25 | POM | 4-trifluoromethylphenyl |
| 26 | POM | 1-naphthyl |
| 27 | POM | 4-chlorophenyl |
| 28 | POM | 3-chlorophenyl |
| 29 | POM | 2-naphthyl |
| 30 | POM | 4-t-butylphenyl |
| 31 | POM | 3-trifluoromethylphenyl |
| 32 | POM | 4-pyridinyl |
| 33 | POM | 3,5-dichlorophenyl |
| 34 | POM | 3,4-dichlorophenyl |
| 35 | POM | 4-methoxyphenyl |
| 36 | POM | 4-biphenyl |
| 37 | BOM | phenyl |
| 38 | BOM | 4-thiomethylphenyl |
| 39 | BOM | 3-flurophenyl |
| 40 | BOM | 4-fluorophenyl |
| 41 | BOM | 4-methylphenyl |
| 42 | BOM | 4-trifluoromethylphenyl |
| 43 | BOM | 1-naphthyl |
| 44 | BOM | 4-chlorophenyl |
| 45 | BOM | 3-chlorophenyl |
| 46 | BOM | 2-naphthyl |
| 47 | BOM | 4-t-butylphenyl |
| 48 | BOM | 3-trifluoromethylphenyl |
| 49 | BOM | 4-pyridinyl |
| 50 | BOM | 3,5-dichlorophenyl |
| 51 | BOM | 3,4-dichlorophenyl |
| 52 | BOM | 4-methoxyphenyl |
| 53 | BOM | 4-biphenyl |
| 54 | NH₄ | 4-thiomethylphenyl |
| 55 | NH₄ | 3-flurophenyl |
| 56 | NH₄ | 4-fluorophenyl |
| 57 | NH₄ | 4-methylphenyl |
| 58 | NH₄ | 4-trifluoromethylphenyl |
| 59 | NH₄ | 1-naphthyl |
| 60 | NH₄ | 4-chlorophenyl |
| 61 | NH₄ | 3-chlorophenyl |
| 62 | NH₄ | 2-naphthyl |
| 63 | NH₄ | 4-t-butylphenyl |
| 64 | NH₄ | 3-trifluoromethylphenyl |
| 65 | NH₄ | 4-pyridinyl |
| 66 | NH₄ | 3,5-dichlorophenyl |
| 67 | NH₄ | 3,4-dichlorophenyl |
| 68 | NH₄ | 4-methoxyphenyl |

or a tautomer thereof, stereoisomer thereof, prodrug thereof, or pharmaceutically acceptable salt thereof.

18. A pharmaceutical composition comprising the compound of claim 1, and a pharmaceutically acceptable excipient.

19-26. (canceled)

27. A herbicide comprising a compound according to claim 1.

28. The compound according to claim 1, wherein
one R₁ is H, C₁₋₆ alkyl, C₁₋₆ haloalkyl, C₃₋₆ cycloalkyl, —(CR^{a}R^{b})ₘ-aryl, —(CR^{a}R^{b})ₘ—O(C═O)—C₁₋₆ alkyl (e.g., —CH₂—O—C(═O)—C(CH₃)₃), —(CR^{a}R^{b})ₘ—O(C═O)—C₃₋₆ cycloalkyl, —(CR^{a}R^{b})ₘ—O(C═O)-aryl (e.g., —CH₂—O—C(═O)—C₆H₅), —(CR^{a}R^{b})ₘ—O(C═O)O—C₁₋₆ alkyl, or —(CR^{a}R^{b})ₘ—O(C═O)O—C₃₋₆ cycloalkyl, —NH₄, —N(alkyl)₄ or —N(aryl)₄, wherein the atom at the left is attached to the oxygen atom; and
the other R₁ is C₁₋₆ alkyl, C₁₋₆ haloalkyl, C₃₋₆ cycloalkyl, —(CR^{a}R^{b})ₘ-aryl, —(CR^{a}R^{b})ₘ—O(C═O)—C₁₋₆ alkyl (e.g., —CH₂—O—C(═O)—C(CH₃)₃), —(CR^{a}R^{b})ₘ—O(C═O)—C₃₋₆ cycloalkyl, —(CR^{a}R^{b})ₘ—O(C═O)-aryl (e.g., —CH₂—O—C(═O)—C₆H₅), —(CR^{a}R^{b})ₘ—O(C═O)O—C₁₋₆ alkyl, or —(CR^{a}R^{b})ₘ—O(C═O)O—C₃₋₆ cycloalkyl, —NH₄, —N(alkyl)₄ or —N(aryl)₄, wherein the atom at the left is attached to the oxygen atom.
